(19) United States
(12) Patent Application Publication
(10) Pub. No.: US 2021/0322590 A1
(43) Pub. Date: Oct. 21, 2021

(54) MOBILE DEVICE FOR DISINFECTING AND STERILIZING

(71) Applicant: TECO Electric & Machinery Co., Ltd., Taipei City (TW)

(72) Inventors: Chia-Jen LIN, Taipei City (TW); Shih-Chang CHEU, Taipei City (TW); Ting-Chang WU, Taipei City (TW)

(21) Appl. No.: 16/918,731

(22) Filed: Jul. 1, 2020

(30) Foreign Application Priority Data
Apr. 20, 2020 (TW) 109113200

(51) Int. Cl. A61L 2/10; A61L 2/24

(52) U.S. Cl. CPC A61L 2/10; A61L 2/24; A61L 2202/25; A61L 2202/14; A61L 2202/16; A61L 2202/11

(57) ABSTRACT

A mobile device includes a mobile platform, an ultraviolet light-emitting module, a scanning module and a controlling module. The mobile platform is used for moving within a working area. The ultraviolet light-emitting module, disposed on an external exposed surface of the mobile platform, is used for generating at least one ultraviolet beam to perform a disinfecting and sterilizing operation within the working area. The scanning module, disposed at the mobile platform and defined with an entire scan range, is used for generating a warning signal upon when a human within the entire scan range is determined. The controlling module, electrically connected with the scanning module and the ultraviolet light-emitting module, is used for controlling the ultraviolet light-emitting module to stop generating the at least one ultraviolet beam upon when the warning signal is received, so that the at least one ultraviolet beam can be prevented from harming the human.

This application claims the benefit of Taiwan Patent Application Serial No. 109113200, filed Apr. 20, 2020, the subject matter of which is incorporated herein by reference.

### BACKGROUND OF THE INVENTION

### (1) Field of the Invention

The invention relates to a device, and more particularly to a mobile device for disinfecting and sterilizing.

### (2) Description of the Prior Art

Tuberculosis, atypical pneumonia (Severe acute respiratory syndrome, SARS), middle east respiratory syndrome (MERS) and other major infections are mainly transmitted through droplets or direct contact.

Wearing a medical mask can substantially avoid droplet infection. However, in addition to washing hands frequently or avoiding direct contact with eyes, mouth and nose, the more important attitude is to perform thorough environmental disinfecting and sterilizing, such that most contagious situations can be resolved.

Currently, disinfecting and sterilizing upon an infected zone are usually carried out by cleaning personnel wearing protective equipment. However, with human labors to perform disinfecting and sterilizing, a risk of infecting virus is always true. Thus, a room is always there for introducing improved environmental disinfecting and sterilizing.

### SUMMARY OF THE INVENTION

In view that a risk of getting infection does always exist while the disinfecting and sterilizing is conducted by human, accordingly it is an object of the present invention to provide a mobile device for disinfecting and sterilizing that can be used for resolving at least one of the aforesaid shortcomings in the art.

In this invention, a mobile device for disinfecting and sterilizing includes a mobile platform, an ultraviolet light-emitting module, a scanning module and a controlling module.

The mobile platform is used for moving within a working area. The ultraviolet light-emitting module, disposed on an external exposed surface of the mobile platform, is used for generating at least one ultraviolet beam to perform a disinfecting and sterilizing operation within the working area. The scanning module, disposed at the mobile platform and defined with an entire scan range, is used for generating a warning signal upon when a human within the entire scan range is determined. The controlling module, electrically connected with the scanning module and the ultraviolet light-emitting module, is used for controlling the ultraviolet light-emitting module to stop generating the at least one ultraviolet beam upon when the warning signal is received, so that the at least one ultraviolet beam can be prevented from harming the human.

In one embodiment of the present invention, the ultraviolet light-emitting module is disposed on a top surface of the external exposed surface of the mobile platform.

In one embodiment of the present invention, the ultraviolet light-emitting module includes a plurality of ultraviolet emission units for generating a plurality of ultraviolet beams including the at least one ultraviolet beam, and at least two of the plurality of ultraviolet beams radiate toward two different directions.

In one embodiment of the present invention, one of the plurality of ultraviolet emission units is an ultraviolet LED light tube for generating the ultraviolet beam having a wavelength ranging between 260 nm and 280 nm.

In one embodiment of the present invention, the scanning module includes a first scan unit and a second scan unit. The first scan for scanning toward a first direction has a first partial scan range defined by two first scan boundaries. The second scan unit for scanning toward a second direction reverse to the first direction has a second partial scan range defined by two second scan boundaries. The two first scan boundaries are located within the second partial scan range, the two second scan boundaries are located within the first partial scan range, and the first partial scan range integrates the second partial scan range to form the entire scan range for covering surroundings of the mobile platform.

In one embodiment of the present invention, the mobile device for disinfecting and sterilizing further includes a path-generating module. The path-generating module electrically connected with the controlling module is used for generating a patrol route with respect to the working area, and the controlling module is used for controlling the mobile platform in a patrol mode to move along the patrol route and further for controlling the ultraviolet light-emitting module to generate the at least one ultraviolet beam.

In one embodiment of the present invention, the mobile device for disinfecting and sterilizing further includes a stop-point generating module. The stop-point generating module electrically connected with the controlling module is used for generating at least one stop point with respect to the working area, and the controlling module is used for controlling the mobile platform in a stop-point mode to move to the at least one stop point and further for controlling the ultraviolet light-emitting module to generate the at least one ultraviolet beam at the at least one stop point.

In one embodiment of the present invention, the mobile device for disinfecting and sterilizing further includes an additional light-emitting module disposed at the mobile platform for generating at least one additional ultraviolet beam. The at least one additional ultraviolet beam is used for radiating a ground surface within the working area so as to perform an additional disinfecting and sterilizing operation to the ground surface.

In one embodiment of the present invention, the additional light-emitting module is disposed on a bottom surface of the mobile platform.

In one embodiment of the present invention, the controlling module includes an additional control unit electrically connected with the additional light-emitting module for controlling the additional light-emitting module to stop generating the at least one additional ultraviolet beam upon when the warning signal is received.

As stated, the mobile device for disinfecting and sterilizing provided by this invention utilizes the ultraviolet light-emitting module of the mobile platform moving within the working area to perform the disinfecting and sterilizing operations. Thereupon, majority human labors can be replaced, and the risk of disease infection can be significantly reduced. In addition, the present invention further introduces the scanning module and the controlling module to turn off the ultraviolet light-emitting module upon when human in the entire scan range is determined, such that the ultraviolet beams can be away from harming the human.

All these objects are achieved by the mobile device for disinfecting and sterilizing described below.

### BRIEF DESCRIPTION OF THE DRAWINGS

The present invention will now be specified with reference to its preferred embodiment illustrated in the drawings, in which:

FIG. 1 is a schematic block view of a preferred embodiment of the mobile device for disinfecting and sterilizing in accordance with this invention;

FIG. 2 is a schematic perspective view of FIG. 1;

FIG. 3 is a schematic side view of FIG. 2 furnished with ultraviolet beams;

FIG. 4 is a schematic top view of FIG. 2 furnished with entire scan ranges;

FIG. 5 is a schematic view showing the preferred mobile device for disinfecting and sterilizing provided by the present invention is performing a disinfecting and sterilizing operation;

FIG. 6 demonstrates schematically the preferred mobile device for disinfecting and sterilizing provided by the present invention is scanning a human;

FIG. 7 shows schematically another scene the preferred mobile device for disinfecting and sterilizing provided by the present invention is scanning a human;

FIG. 8 demonstrates schematically the mobile device for disinfecting and sterilizing provided by the present invention is in a patrol mode; and

FIG. 9 demonstrates schematically the mobile device for disinfecting and sterilizing provided by the present invention is in a stop-point mode.

### DESCRIPTION OF THE PREFERRED EMBODIMENT

The invention disclosed herein is directed to a mobile device for disinfecting and sterilizing. In the following description, numerous details are set forth in order to provide a thorough understanding of the present invention. It will be appreciated by one skilled in the art that variations of these specific details are possible while still achieving the results of the present invention. In other instance, well-known components are not described in detail in order not to unnecessarily obscure the present invention.

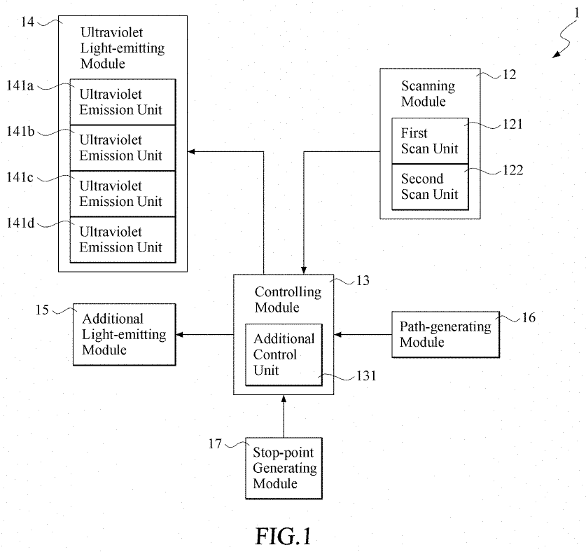

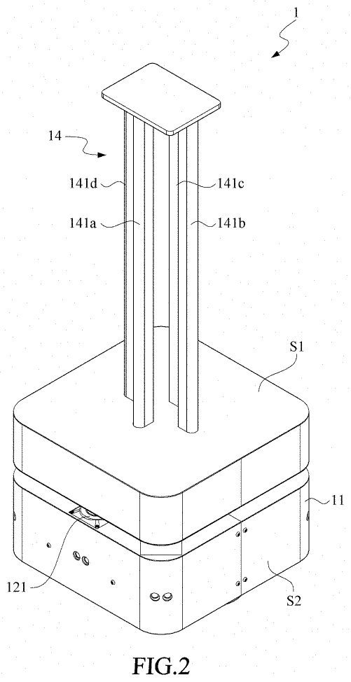

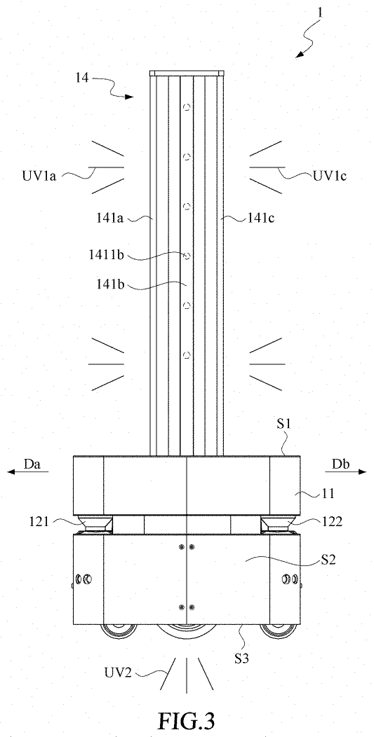

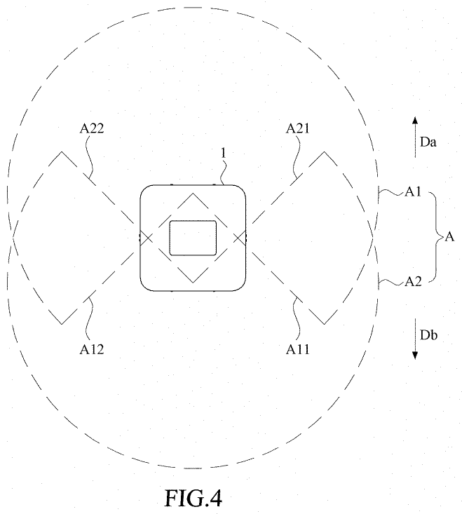

Refer to FIG. 1 through FIG. 4; where FIG. 1 is a schematic block view of a preferred embodiment of the mobile device for disinfecting and sterilizing in accordance with this invention, FIG. 2 is a schematic perspective view of FIG. 1, FIG. 3 is a schematic side view of FIG. 2 furnished with ultraviolet beams, and FIG. 4 is a schematic top view of FIG. 2 furnished with entire scan ranges. As shown, the preferred mobile device for disinfecting and sterilizing (hereinafter being simplified as “mobile device” in the following preferred embodiment) 1 includes a mobile platform 11, a scanning module 12, a controlling module 13, an ultraviolet light-emitting module 14, an additional light-emitting module 15, a path-generating module 16 and a stop-point generating module 17.

The ultraviolet light-emitting module 14, disposed on an external exposed surface of the mobile platform 11, is used for generating at least one ultraviolet beam. In this embodiment, the external exposed surface can be, but not limited to, a top surface S1. The external exposed surface, directed to an exposed surface of the mobile platform 11, includes a top surface S1 or a lateral surface S2.

In this embodiment, the ultraviolet light-emitting module 14 includes four ultraviolet emission units 141a, 141b, 141c, 141d for individually and independently generating ultraviolet beams UV1a, UV1b, UV1c, UV1d, respectively. In particular, all the ultraviolet beams UV1a, UV1b, UV1c, UV1d have different radiating directions. Wavelengths of the ultraviolet beams UV1a, UV1b, UV1c, UV1d are ranged between 260 nm and 280 nm.

Practically, each of the ultraviolet emission units 141a, 141b, 141c, 141d can adopt an ultraviolet LED (UVC LED) light tube. For example, as shown in FIG. 3, the ultraviolet emission unit 141b is embodied as a UVC LED light tube including a plurality of LED chips (six shown in the figure, but only one labeled by 1411b). Since the LED light tube including LED chips is well known in the art, thus details thereabout would be omitted herein. In addition, the ultraviolet emission unit 141a, 141b, 141c or 141d can adopt an ultraviolet fluorescent light tube. Nevertheless, no matter what a UVC LED light tube or an ultraviolet fluorescent light tube is, each of the ultraviolet emission units 141a, 141b, 141c, 141d generally includes a Quartz glass able to transmit light and to enclose an inner tube space.

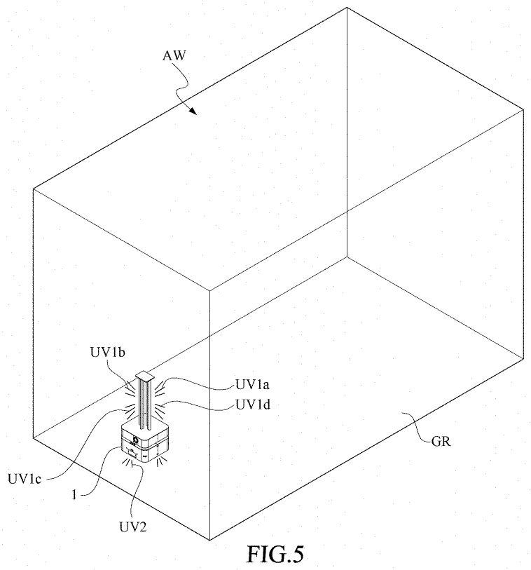

The additional light-emitting module 15, disposed on a bottom surface S3 of the mobile platform 11, is used for generating at least one additional ultraviolet beam UV2 having a radiation direction different that of the ultraviolet beam UV1a, UV1b (labeled in FIG. 5), UV1c or UV1d (labeled in FIG. 5).

The scanning module 12, disposed at the mobile platform 11, has an entire scan range A, and includes a first scan unit 121 and a second scan unit 122. The first scan unit 121, for scanning toward a first direction Da, has a first partial scan range A1 defined by two first scan boundaries A11, A12. The second scan unit 122, for scanning toward a second direction Db, has a second partial scan range A2 defined by two second scan boundaries A21, A22. In this embodiment, the first direction Da and the second direction Db are reverse directions.

In this embodiment, the first scan unit 121 and the second scan unit 122 of the scanning module 12 are disposed in front and rear sides of the mobile platform 11, respectively, and can (but not limited to) be fitted into the mobile platform 11. The scanning module 12 can be disposed to some other positions at the mobile platform 11, such as the top surface S1 of the mobile platform 11, each of four corners of the mobile platform 11 and the front or rear side of the mobile platform 11 (without being fitted into the mobile platform 11).

Further, since the first scan boundaries A11, A12 are extended within the second partial scan range A2 while the second scan boundaries A21, A22 are within the first partial scan range A1, thus the entire scan range A integrally formed by the first partial scan range A1 and the second partial scan range A2 would cover the surroundings of the mobile platform 11; namely, all the surroundings of the mobile device 1.

The controlling module 13 is electrically connected with the scanning module 12, the ultraviolet light-emitting module 14, the additional light-emitting module 15, the path-generating module 16 and the stop-point generating module 17.

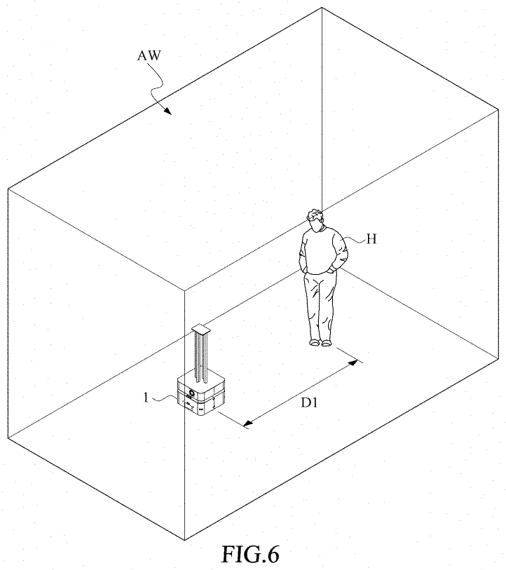

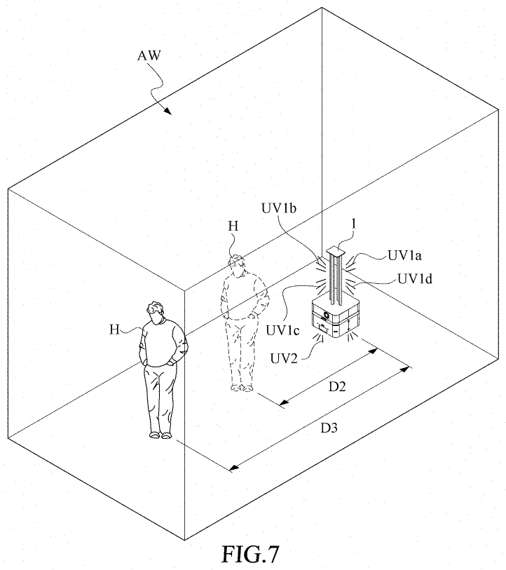

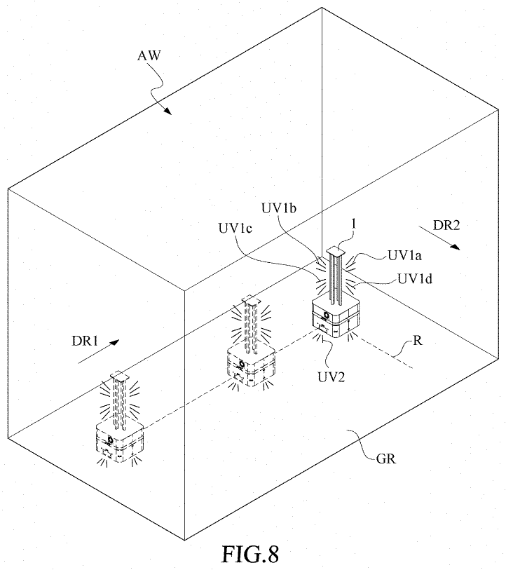

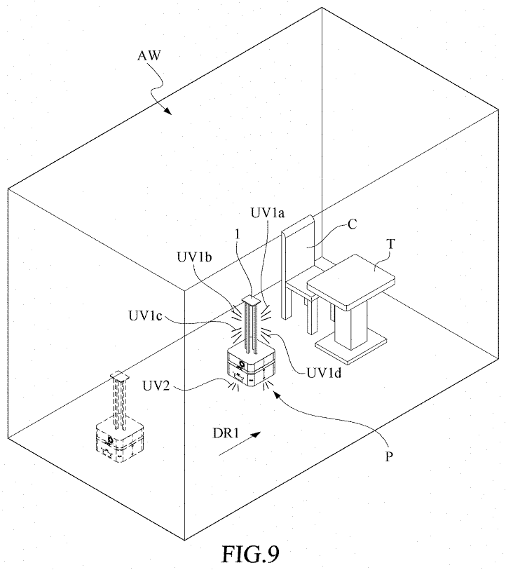

Then, refer now together from FIG. 1 to FIG. 9; where FIG. 5 is a schematic view showing the preferred mobile device for disinfecting and sterilizing provided by the present invention is performing a disinfecting and sterilizing operation, FIG. 6 demonstrates schematically the preferred mobile device for disinfecting and sterilizing provided by the present invention is scanning a human, FIG. 7 shows schematically another scene the preferred mobile device for disinfecting and sterilizing provided by the present invention is scanning a human, FIG. 8 demonstrates schematically the mobile device for disinfecting and sterilizing provided by the present invention is in a patrol mode, and FIG. 9 demonstrates schematically the mobile device for disinfecting and sterilizing provided by the present invention is in a stop-point mode. As shown, the mobile device for disinfecting and sterilizing 1 can move within a working area AW. The working area AW can be a hospital, a classroom, a building, a department store, a cafeteria, or any place that requires disinfecting and sterilizing.

The ultraviolet emission units 141a, 141b, 141c, 141d can individually generate the ultraviolet beams UV1a, UV1b, UV1c, UV1d, respectively, for performing a disinfecting and sterilizing operation in the working area AW. Since the ultraviolet beams UV1a, UV1b, UV1c, UV1d are arranged to radiate toward different directions, thus the maximum disinfecting and sterilizing area can be obtained.

Since the ultraviolet emission units 141a, 141b, 141c, 141d are all disposed on the top surface S1, the beams for disinfecting and sterilizing may not reach a portion under the mobile device for disinfecting and sterilizing 1 or a ground surface GR in the working area AW. Hence, the additional ultraviolet beams UV2 generated by the additional light-emitting module 15 can be introduced to radiate the ground surface GR, such that an additional disinfecting and sterilizing operation can be performed on the ground surface GR.

The mobile device for disinfecting and sterilizing 1 can utilize the ultraviolet light-emitting module 14 and the additional light-emitting module 15 to perform the disinfecting and sterilizing operation and the additional disinfecting and sterilizing operation over the entire working area AW, as shown in FIG. 5. Thereupon, the mobile device for disinfecting and sterilizing 1 provided by the present invention can successfully replace human labors for performing the disinfecting and sterilizing operations, and thereby various problems caused by human work can be substantially resolved.

In this embodiment, since the ultraviolet beams UV1a, UV1b, UV1c, UV1d and the additional ultraviolet beams UV2 are all ultraviolet, thus a long-term radiation would harm the human. Thus, whenever the scanning module 12 scans and determines that a human H exists within the entire scan range A, a corresponding warning signal would be generated. Upon when the controlling module 13 receives the warning signal, the ultraviolet light-emitting module 14 would be controlled to close, such that the ultraviolet light-emitting module 14 would stop generating the ultraviolet beams UV1a, UV1b, UV1c, UV1d, and so that the radiation of the ultraviolet beams UV1a, UV1b, UV1c, UV1d upon the human H can be avoided. Preferably, the controlling module 13 includes an additional control unit 131. As soon as the additional control unit 131 receives the warning signal, the additional light-emitting module 15 would be turned off simultaneously so as to have the additional light-emitting module 15 not to generate the additional ultraviolet beams UV2.

Further, as shown in FIG. 6, a human H is spaced from the mobile device for disinfecting and sterilizing 1 by a distance D1. When the scanning module 12 scans and determines a human H in the entire scan range A, a warning signal would be generated. Then, the controlling module 13 would turn off the ultraviolet light-emitting module 14 and the additional light-emitting module 15. At the same time, the mobile device for disinfecting and sterilizing 1 will not generate any ultraviolet beam UV1a, UV1b, UV1c or UV1d, or the additional ultraviolet beam UV2, as shown in FIG. 6.

When the human H keeps walking to space from the mobile device for disinfecting and sterilizing 1 by a distance D3, and then if the scan of the scanning module 12 would determine that the human H has now already left the entire scan range A, the controlling module 13 would restart the ultraviolet light-emitting module 14 and the additional light-emitting module 15 so as to have the ultraviolet light-emitting module 14 and the additional light-emitting module 15 to resume the generation of the ultraviolet beams UV1a, UV1b, UV1c, UV1d and the additional ultraviolet beams UV2. It shall be explained that, when the human H moves to a place behind the mobile device 1 by a distance D2 (symbolized by the dashed lines in the figure) but when the scanning module 12 scans to determine that the human H is still in the entire scan range A, then the controlling module 13 will not restart the ultraviolet light-emitting module 14 and the additional light-emitting module 15.

Therefore, the mobile device 1 can prevent the ultraviolet beams UV1a, UV1b, UV1c, UV1d and the additional ultraviolet beams UV2 from radiating the human H in a long term, such that possible harm to the human H can be avoided.

In this embodiment, the ultraviolet emission units 141a, 141b, 141c, 141d can be, but not limited to, digital driven ultraviolet lamps. In another embodiment, the ultraviolet emission units 141a, 141b, 141c, 141d can be analog ultraviolet lamps. The merit of the digital driven ultraviolet lamp is that the performance in power supply and stabilizing the voltage would be better. In addition, the possibility of device damage from frequently turning on/off would be lower in the digital driven ultraviolet lamp than the analog ultraviolet lamp. In addition, a driving voltage of the digital driven ultraviolet lamp is 26V, smaller than the 110-V driving voltage for the analog ultraviolet lamp.

The path-generating module 16 is used for generating a patrol route R with respect to the working area AW. The controlling module 13 would control the mobile platform 11 to move along the patrol route R, and control the ultraviolet light-emitting module 14 and the additional light-emitting module 15 to generate the ultraviolet beams UV1a, UV1b, UV1c, UV1d and the additional ultraviolet beams UV2.

As shown in FIG. 8, the controlling module 13 controls the mobile platform 11 to move along the patrol route R, firstly in a moving direction DR1 and then in a moving direction DR2. In FIG. 8, dashed lines are used to indicate previous positions of the mobile device 1. Thus, it is shown that, as the mobile device 1 moves along the patrol route R, both the ultraviolet light-emitting module 14 and the additional light-emitting module 15 would generate the ultraviolet beams UV1a, UV1b, UV1c, UV1d and the additional ultraviolet beams UV2. In other words, the mobile device 1 performs the disinfecting and sterilizing operation and the additional disinfecting and sterilizing operation at the same time while moving in the working area AW. The aforesaid patrol mode can be applied to a corridor, a promenade, a hall and another place with few obstacles or more space.

It shall be explained that, practically, the patrol route R is not a real path or a real surrounding road. Actually, the patrol route R is only a scheme in the working area AW, and thus plotted by dashed lines.

The stop-point generating module 17 is used for generating at least one stop point in correspondence with the working area AW. The controlling module 13 would move the mobile platform 11 to each of the stop points. Whenever the mobile device 1 arrives at each of the stop points, the ultraviolet light-emitting module 14 and the additional light-emitting module 15 would be controlled to generate the ultraviolet beams UV1a, UV1b, UV1c, UV1d and the additional ultraviolet beams UV2. In this embodiment, only one stop point is included and labeled as “P” in the figure. However, in some other embodiments of this invention, the quantity of the stop points within the working area AW may be a plural number.

As shown in FIG. 9, the controlling module 13 controls the mobile platform 11 to move in the moving direction DR1 to reach the stop point P. In FIG. 9, the dashed-line mobile device 1 demonstrates a previous position of the mobile device 1. Thus, it is easy to observe that, while the mobile device 1 follows the moving direction DR1 to move forward, the ultraviolet light-emitting module 14 and the additional light-emitting module 15 would not generate the ultraviolet beams UV1a, UV1b, UV1c, UV1d and the additional ultraviolet beams UV2. Till when the mobile device 1 reaches the stop point P, then the controlling module 13 begins to control the ultraviolet light-emitting module 14 and the additional light-emitting module 15 to generate the ultraviolet beams UV1a, UV1b, UV1c, UV1d and additional ultraviolet beams UV2.

In this embodiment, the stop point P is at a position respective to a table T and a chair C. After the controlling module 13 controls the mobile device 1 moving to the stop point P, then the disinfecting and sterilizing operation can be performed onto the table T and the chair C. The aforesaid stop-point mode can be applied to specific places that require special arrangement of disinfecting and sterilizing, such as footprints of people diagnosed to confirm the disease, touched things of these people, or potential places easy to accumulate viruses.

In summary, the mobile device for disinfecting and sterilizing provided by this invention utilizes the ultraviolet light-emitting module of the mobile platform moving within the working area to perform the disinfecting and sterilizing operations. Thereupon, majority human labors can be replaced, and the risk of disease infection can be significantly reduced. In addition, the present invention further introduces the scanning module and the controlling module to turn off the ultraviolet light-emitting module upon when human in the entire scan range is determined, such that the ultraviolet beams can be away from harming the human.

While the present invention has been particularly shown and described with reference to a preferred embodiment, it will be understood by those skilled in the art that various changes in form and detail may be without departing from the spirit and scope of the present invention.

What is claimed is:

1. A mobile device for disinfecting and sterilizing, comprising:
a mobile platform, used for moving within a working area;
an ultraviolet light-emitting module, disposed on an external exposed surface of the mobile platform, used for generating at least one ultraviolet beam to perform a disinfecting and sterilizing operation within the working area;
a scanning module, disposed at the mobile platform, defined with an entire scan range, used for generating a warning signal upon when a human within the entire scan range is determined; and
a controlling module, electrically connected with the scanning module and the ultraviolet light-emitting module, used for controlling the ultraviolet light-emitting module to stop generating the at least one ultraviolet beam upon when the warning signal is received, so as to prevent the at least one ultraviolet beam from harming the human.

2. The mobile device for disinfecting and sterilizing of claim 1, wherein the ultraviolet light-emitting module is disposed on a top surface of the external exposed surface of the mobile platform.

3. The mobile device for disinfecting and sterilizing of claim 1, wherein the ultraviolet light-emitting module includes a plurality of ultraviolet emission units for generating a plurality of ultraviolet beams including the at least one ultraviolet beam, and at least two of the plurality of ultraviolet beams radiate toward two different directions.

4. The mobile device for disinfecting and sterilizing of claim 3, wherein one of the plurality of ultraviolet emission units is an ultraviolet LED light tube for generating the ultraviolet beam having a wavelength ranging between 260 nm and 280 nm.

5. The mobile device for disinfecting and sterilizing of claim 1, wherein the scanning module includes:
a first scan unit, scanning toward a first direction, having a first partial scan range defined by two first scan boundaries; and
a second scan unit, scanning toward a second direction reverse to the first direction, having a second partial scan range defined by two second scan boundaries;
wherein the two first scan boundaries are located within the second partial scan range, the two second scan boundaries are located within the first partial scan range, and the first partial scan range integrates the second partial scan range to form the entire scan range for covering surroundings of the mobile platform.

6. The mobile device for disinfecting and sterilizing of claim 1, further including a path-generating module, wherein the path-generating module electrically connected with the controlling module is used for generating a patrol route with respect to the working area, and the controlling module is used for controlling the mobile platform in a patrol mode to move along the patrol route and further for controlling the ultraviolet light-emitting module to generate the at least one ultraviolet beam.

7. The mobile device for disinfecting and sterilizing of claim 1, further including a stop-point generating module, wherein the stop-point generating module electrically connected with the controlling module is used for generating at least one stop point with respect to the working area, and the controlling module is used for controlling the mobile platform in a stop-point mode to move to the at least one stop point and further for controlling the ultraviolet light-emitting module to generate the at least one ultraviolet beam at the at least one stop point.

8. The mobile device for disinfecting and sterilizing of claim 1, further including an additional light-emitting module disposed at the mobile platform for generating at least one additional ultraviolet beam, wherein the at least one additional ultraviolet beam is used for radiating a ground surface within the working area so as to perform an additional disinfecting and sterilizing operation to the ground surface.

9. The mobile device for disinfecting and sterilizing of claim 8, wherein the additional light-emitting module is disposed on a bottom surface of the mobile platform.

10. The mobile device for disinfecting and sterilizing of claim 8, wherein the controlling module includes an additional control unit electrically connected with the additional light-emitting module for controlling the additional light-emitting module to stop generating the at least one additional ultraviolet beam upon when the warning signal is received.
